(19) United States
(12) Patent Application Publication
(10) Pub. No.: US 2010/0292536 A1
(43) Pub. Date: Nov. 18, 2010

(54) FLUID CONTROL DEVICE AND FLUID CONTROL METHOD, AND ENDOSCOPE APPARATUS

(76) Inventors: Shinichi Yamakawa, Ashigarakami-gun (JP); Tsuyoshi Ashida, Ashigarakami-gun (JP); Takayuki Nakamura, Ashigarakami-gun (JP)

Correspondence Address: BIRCH STEWART KOLASCH & BIRCH, PO BOX 747, FALLS CHURCH, VA, 22040-0747, US

(21) Appl. No.: 12/779,123

(22) Filed: May 13, 2010

(30) Foreign Application Priority Data
May 14, 2009 (JP) 2009-117656

(51) Int. Cl. A61B 1/00; A61M 29/00

(52) U.S. Cl. 600/116; 604/99.01; 604/100.01

(57) ABSTRACT

A fluid control device which controls a flow rate of a fluid supplied or sucked to or from an inflation and deflation member, includes: a first pipe system connected to the inflation and deflation member, wherein the fluid flow of a predetermined flow rate is supplied to the first pipe system; a second pipe system connected to the inflation and deflation member, wherein a fluid flow of a flow rate lower than the predetermined flow rate is supplied to the second pipe system; a device which switches the first pipe system to the second pipe system when a predetermined amount of fluid is supplied or sucked to or from the inflation and deflation member through the first pipe system; and a device which controls the pressure in the inflation and deflation device by using the second pipe system based on a pressure in the second pipe system.

### CROSS-REFERENCE TO RELATED APPLICATIONS

This application claims the priority benefit under 35 U.S.C. §119 of Japanese Patent Application No. 2009-117656 filed on May 14, 2009, which is hereby incorporated in its entirety by reference.

### BACKGROUND OF THE INVENTION

1. Field of the Invention

The presently disclosed subject matter relates to a fluid control device and a fluid control method, and an endoscope apparatus, and particularly relates to an art of protecting a pressure sensor (for example, a pressure sensor with high precision such as a pressure sensor used for the purpose of performing pressure control after application of a high pressure to a conduit line in order to inflate an inflation and deflation member such as a balloon of an endoscope at a high speed) from a high pressure.

2. Description of the Related Art

A large intestine has an anfractuous (curvy) structure inside a body, and has a part which is not fixed to a body cavity. Then, it is difficult to insert an endoscope into a large intestine. Therefore, much experience is required to acquire a technique to insert an endoscope into a large intestine. If an operator does not have much experience, the operator may inflict much pain to a patient.

In order to assist in insertion of an endoscope into a body, an endoscope and an insertion auxiliary tool using balloons have been developed. For example, an endoscope with a balloon is known, in which the balloon which is inflated by air supply and is deflated by air exhaust is provided at an endoscope insertion portion or an insertion auxiliary tool. By using the endoscope with a balloon, the endoscope insertion portion inserted into a body cavity, or the insertion auxiliary tool which allows the endoscope insertion portion to be inserted through and guided can be fixed to an inside of a body cavity.

Inflation/deflation of these balloons requires five to ten seconds. However, in order to enhance the performance of surgery, it is necessary to inflate and deflate the balloons more quickly. In order to shorten the inflating/deflating time, it is necessary to increase the flow rate (enhance a flow velocity) of a fluid (air). Meanwhile, a conduit line leading to the balloon has an inside diameter of about 1 to 2 millimeters, and is very thin. In order to pass a large flow into the conduit line, a large pressure difference is required at the distal end side and the proximal end side of the conduit line. More specifically, the fluid at the processor side (proximal end side) needs to be at a high pressure.

In regard to this, the endoscopes and the insertion auxiliary tools using the balloons as in Japanese Patent Application Laid-Open No. 2005-261782, Japanese Patent Application Laid-Open No. 2007-185370, and Japanese Patent Application Laid-Open No. 2007-209626 are proposed.

First, Japanese Patent Application Laid-Open No. 2005-261782 discloses an endoscope system including a fixing balloon attached to an outer peripheral portion of a distal end of an insertion portion of an endoscope, and a tube fixing balloon attached to an outer peripheral portion of a distal end of an over tube which allows the endoscope to be inserted therethrough and guides the insertion portion when the insertion portion is inserted into, for example, an alimentary canal. The endoscope system disclosed in Japanese Patent Application Laid-Open No. 2005-261782 delivers a fixed amount by total flow rate detection by a timer while confirming that a pump side pressure is within a predetermined range.

Further, Japanese Patent Application Laid-Open No. 2007-185370 discloses an endoscope apparatus including a first balloon fixed to the outer peripheral surface of an insertion portion of an endoscope to be inserted into a body cavity, and a second balloon fixed in the vicinity of the distal end of the insertion auxiliary tool having the inside diameter which is slightly larger than the outside diameter of the insertion portion. In the endoscope apparatus of Japanese Patent Application Laid-Open No. 2007-185370, both the pressure sensor and the flow rate sensor are provided on a single conduit line so that the supply and exhaust amounts are controlled in accordance with a pressure.

Further, Japanese Patent Application No. 2007-209626 discloses an endoscope apparatus including a first balloon fixed to the outer peripheral surface of an insertion portion of an endoscope and a second balloon fixed in the vicinity of the distal end of an insertion auxiliary tool. The endoscope apparatus of Japanese Patent Application No. 2007-209626 has a flow rate regulating device which regulates supply and exhaust amounts, so that the fluid is allowed to flow at a high flow rate first, and after the pressure reaches a certain pressure, the fluid is allowed to flow at a low flow rate.

As above, in these proposals, the pressure sensor, the flow rate control device, and the flow rate sensor are disposed each in the single conduit line which connects the pump and the balloon, and enhancement in flow velocity can be realized by switching the flow rate control and pressure control.

### SUMMARY OF THE INVENTION

However, when enhancing the flow velocity of the fluid in the conduit line, the inside of the conduit line at the processor side can be under a high pressure. Therefore, the pressure sensor disposed in the conduit line needs to have a wide dynamic range. Also, since the balloon internal pressure is kept within a certain range (for example, about 5 kPa) to control the balloon safely, and the pressure sensor with high precision with a narrow dynamic range is required. Accordingly, a large flow amount of fluid cannot be supplied to the conduit line if the pressure sensor with high precision is used in order to control the balloon internal pressure accurately, and proper fluid control cannot be performed.

The presently disclosed subject matter is made in view of such circumstances, and has an object to provide a fluid control device and a fluid control method, and an endoscope apparatus which can perform proper fluid control by using a pressure sensor with high precision while supplying a fluid with a flow velocity to a inflation and deflation member for the endoscope apparatus.

In order to attain the above described object, a first aspect of the presently disclosed subject matter provides A fluid control device which controls a flow rate of a fluid which is supplied or sucked to or from an inflation and deflation member included in an endoscope or an endoscope auxiliary tool, including: a fluid flow generation device which generates a fluid flow to supply or suck the fluid to or from the inflation and deflation member; a first pipe system which is connected to the inflation and deflation member, wherein the fluid flow of a predetermined flow rate generated by the fluid flow generation device is supplied to the first pipe system to supply or suck the fluid to or from the inflation and deflation member; a second pipe system which is connected to the inflation and deflation member, wherein a fluid flow of a flow rate lower than the predetermined flow rate is supplied to the second pipe system to supply or suck the fluid to or from the inflation and deflation member; a pipe system switching device which switches the first pipe system to the second pipe system when a predetermined amount of fluid is supplied or sucked to or from the inflation and deflation member through the first pipe system; a pressure sensor which is included in the second pipe system, and measures a pressure in the second pipe system; and a pressure control device which controls the pressure in the inflation and deflation device by using the second pipe system based on the pressure measured by the pressure sensor.

As a result, while a high flow velocity is realized at an initial inflating time or at an initial deflating time of an inflation and deflation member when the fluid flow is supplied to the first pipe system. By switching the first pipe system to second pipe system, fluid control can be performed properly by using the pressure sensor with high precision in the second pipe system.

Further, a second aspect of the presently disclosed subject matter provides a fluid control device according to the first aspect, further including: a leaking device which regulates the flow rate of the fluid by leaking a part of the fluid to an outside; and a main conduit line which allows the fluid flow generation device and the inflation and deflation member to communicate with each other, wherein an output of the fluid flow generation device is constant, the second pipe system is a conduit line which connects the leaking device and the pressure sensor to the main conduit line, and the first pipe system is a conduit line which extends from the fluid flow generation device to the inflation and deflation member by passing a bypass conduit line which bypasses the leaking device and the pressure sensor, and includes a flow rate adjustment device for supplying or sucking a fixed amount of the fluid to or from the inflation and deflation member.

Thereby, when a high flow velocity is realized at the initial inflating time or at the initial deflating time of the inflation and deflation member, the pressure sensor with high precision can be protected by bypassing the pressure sensor.

Further, a third aspect of the presently disclosed subject matter provides a fluid control device according to the first aspect, further including: a leaking device which regulates the flow rate of the fluid by leaking a part of the fluid to an outside; and a main conduit line which allows the fluid flow generation device and the inflation and deflation member to communicate with each other, connects the leaking device and the pressure sensor to be capable of communicating with each other and being cut off from each other, and includes a flow rate adjustment device for supplying or sucking a constant amount of the fluid to or from the inflation and deflation member, wherein the fluid flow generation device has a constant output, the first pipe system is a conduit line for cutting off the leaking device and the pressure sensor from the main conduit line, and the second pipe system is a conduit line which allows the leaking device and the pressure sensor to communicate with the main conduit line.

Thereby, when a high flow velocity is realized at the initial inflating time or at the initial deflating time of the inflation and deflation member, the pressure sensor with high precision can be protected by cutting off the pressure sensor from the fluid at a high pressure.

Further, a fourth aspect of the presently disclosed subject matter provides a fluid control device according to the second and third aspect, wherein the flow rate adjustment device is a flow rate integration detecting device which detects that the amount of the fluid which is supplied or sucked to or from the inflation and deflation member reaches a predetermined amount.

Thereby, at the initial inflating time or at the initial deflating time of the inflation and deflation member, a pressure higher than required can be prevented from being exerted on the inflation and deflation member.

Further, a fifth aspect of the presently disclosed subject matter provides a fluid control device according to the fourth aspect, wherein the flow rate integration detecting device includes at least a pressure gauge.

Further, a sixth aspect of the presently disclosed subject matter provides a fluid control device according to the fourth aspect, wherein the flow rate integration detecting device includes at least a differential pressure gauge.

Further, a seventh aspect of the presently disclosed subject matter provides a fluid control device according to the fourth aspect, wherein the flow rate integration detecting device includes at least a flowmeter.

Further, an eighth aspect of the presently disclosed subject matter provides a fluid control device according to the second and third aspect, wherein the flow rate adjustment device is a syringe which supplies or sucks a constant amount of the fluid which is set in advance to or from the inflation and deflation member.

Further, a ninth aspect of the presently disclosed subject matter provides a fluid control device according to the second to eighth aspect, wherein the leaking device is a speed controller. Further, a tenth aspect of the presently disclosed subject matter provides a fluid control device according to the second to eighth aspect, wherein the leaking device is a device for naturally releasing the main conduit line. Further, an eleventh aspect of the presently disclosed subject matter provides a fluid control device according to the second to eighth aspect, wherein the leaking device is a pressure reducing valve.

Thereby, the flow rate can be regulated by combining the fluid flow generating source with a constant output and these leaking devices.

Further, in order to attain the above described object similarly, a twelfth aspect of the presently disclosed subject matter provides a fluid control method for controlling a flow rate of a fluid which is supplied or sucked to or from an inflation and deflation member included in an endoscope or an endoscope auxiliary tool by a fluid flow generation device, including the steps of: generating a fluid flow to supply or suck the fluid to or from the inflation and deflation member; supplying the fluid flow of a predetermined flow rate generated by the fluid flow generation device to a first pipe system connected to the inflation and deflation member, to supply or suck the fluid to or from the inflation and deflation member; switching the first pipe system to a second pipe system when a predetermined amount of fluid is supplied or sucked to or from the inflation and deflation member through the first pipe system; supplying a fluid flow of a flow rate lower than the predetermined flow rate to the second pipe system connected to the inflation and deflation member, to supply or suck the fluid to or from the inflation and deflation member; and controlling the pressure in the inflation and deflation device by controlling the fluid flow in the second pipe system based on the pressure measured by a pressure sensor which measures a pressure in the second pipe system.

As a result, while a high flow velocity is realized at an initial inflating time or at an initial deflating time of an inflation and deflation member, by switching the pipe system, the pressure sensor with high precision is protected, and proper fluid control can be performed by using the pressure sensor with high precision.

Further, in order to attain the above described object similarly, a thirteenth aspect of the presently disclosed subject matter provides an endoscope apparatus including a fluid control device according to any one of the first to the eleventh aspects.

As described above, according to the presently disclosed subject matter, while a high flow velocity is realized at an initial inflating time or at an initial deflating time of an inflation and deflation member, proper fluid control can be performed by using the pressure sensor with high precision by switching a pipe system.

### BRIEF DESCRIPTION OF THE DRAWINGS

FIG. 1 is a schematic configuration diagram illustrating an embodiment of an electronic endoscope including a fluid control device according to the presently disclosed subject matter;

FIG. 2 is an enlarged sectional view along an axial direction (longitudinal direction) of a distal end portion of an insertion portion of the electronic endoscope illustrated in FIG. 1;

FIG. 3 is a block diagram of a balloon control device configured to control pressures of a driving balloon, a locking balloon and a holding balloon;

FIGS. 4A to 4E are enlarged sectional views along the axial direction (longitudinal direction) of the distal end portion of the insertion portion of the electronic endoscope illustrating a state of inflation and deflation of each of the balloons at a time of a normal traveling operation;

FIG. 5 is a block diagram illustrating a first embodiment of a fluid control system of the presently disclosed subject matter;

FIG. 6 is a timing chart illustrating control of each of electromagnetic valves in the case of inflating the balloons in the first embodiment;

FIG. 7 is a block diagram illustrating a second embodiment of the fluid control system of the presently disclosed subject matter;

FIG. 8 is a block diagram illustrating a third embodiment of the fluid control system of the presently disclosed subject matter; and

FIG. 9 is a block diagram illustrating a fourth embodiment of the fluid control system of the presently disclosed subject matter.

### DETAILED DESCRIPTION OF THE PREFERRED EMBODIMENTS

Hereinafter, a fluid control device and a fluid control method, and an endoscope apparatus according to the presently disclosed subject matter will be described in detail with reference to the attached drawings.

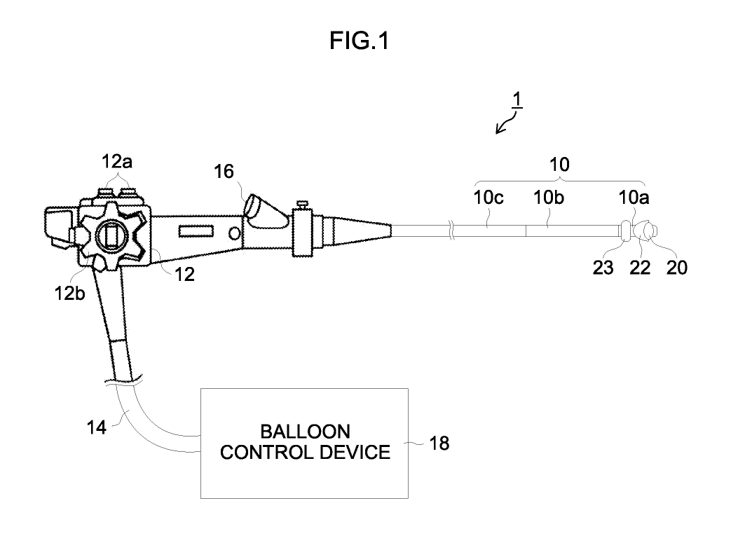

FIG. 1 is a schematic configuration diagram illustrating an embodiment of an electronic endoscope including the fluid control device according to the presently disclosed subject matter.

As shown FIG. 1, an electronic endoscope 1 includes an insertion portion 10 and an operation portion 12 which is connectively provided at a proximal end portion of the insertion portion 10. The insertion portion 10 is a movable body which is inserted into a canal such as an alimentary canal of a subject, and can move in the canal. In a distal end portion 10a which is connectively provided at a distal end of the insertion portion 10, an objective lens (not illustrated) for capturing image light of an observation region in the canal of a subject, and an imaging element (not illustrated) which images the image light are contained. The image of the inside of the canal of the subject is acquired by the imaging element contained in the distal end portion 10a, and is displayed on a monitor of a processor device (not illustrated) connected to a cord 14 as an endoscope image.

Further, the distal end portion 10a includes an illumination window, a forceps outlet port communicating with a forceps port 16 and a nozzle (not illustrated). An illumination light from a light source device (not illustrated) is irradiated onto an observation region through the illumination window. Cleaning water or air for cleaning the contaminants on an observation window which protects the objective lens is sprayed from the nozzle when operating an air supply/water supply button 12a.

A bending portion 10b is provided on the rear side (proximal end side) of the distal end portion 10a. The bending portion 10b includes a plurality of bending pieces which are connected with each other. The bending portion 10b performs a bending operation in a vertical direction and a lateral direction when an operator operates an angle knob 12b provided at the operation portion 12 to push and/or pull a wire inserted in the insertion portion 10. Thereby, the distal end portion 10a can be directed to a desired direction in the canal of the subject.

A flexible portion 10c having flexibility is provided on the rear side (proximal end side) of the bending portion 10b. The flexible portion 10c has a length of one to several meters so that the distal end portion 10a can reach an observation region (ROI; region of interest), and an operator can keep a distance from a subject (patient) to such an extent as not to cause a trouble when an operator grips the operation portion 12 and operates the endoscope 1.

At the distal end portion 10a, a driving balloon 20 and a locking balloon 22 are attached. The driving balloon 20 and the locking balloon 22 are inflation and deflation members. The driving balloon 20 and the locking balloon 22 are disposed and fixed side by side along a traveling direction of the distal end portion 10a moving inside the canal of a subject. The driving balloon 20 and the locking balloon 22 are mainly formed by latex rubber capable of inflating and deflating, and are connected to a balloon control device 18 for controlling the pressures inside the balloons.

In the distal end portion 10a, the driving balloon 20 and the locking balloon 22 are disposed adjacently to each other, and are formed on an entire circumference in the circumferential direction of the insertion portion 10. Further, the driving balloon 20 and the locking balloon 22 may be formed into uniform shapes in the circumferential direction of the insertion portion 10 to be axially symmetrical. The driving balloon 20 and the locking balloon 22 do not have to be in uniform shapes in the circumferential direction, or do not have to be axially symmetrical.

Further, the driving balloon 20 and the locking balloon 22 may be disposed at the bending portion 10b or the flexible portion 10c.

In order to observe an inner wall surface of a canal (for example, an alimentary canal such as a large intestine and a small intestine) which are complicatedly bent by using the electronic endoscope 1, the insertion portion 10 is inserted into a subject with the driving balloon 20 and the locking balloon 22 deflated. When inserting the insertion portion 10 into the subject, the monitor displays an endoscope image obtained by the imaging element while the light source device is lit to illuminate the inside of the subject. Then, the operator can operate the endoscope observing the endoscope image displayed on the monitor.

When the distal end portion 10a reaches the inside of the alimentary canal, the balloon control device 18 controls inflation/deflation of the driving balloon 20 and the locking balloon 22 to act a pressure on the inner wall surface of the alimentary canal. Thereby, the inner wall surface of the alimentary canal is pulled in, and the insertion portion 10 propels forward or backward in the traveling direction relatively to the inner wall surface of the alimentary canal. The details of operation of the balloons 20 and 22 will be described later.

In the following description, the operation of propelling the distal end portion 10a forward in the traveling direction (propelling the distal end portion 10a into a deeper portion of the canal) will be described as a normal traveling operation, and the operation of propelling the distal end portion 10a backward in the traveling direction is described as a reversing operation.

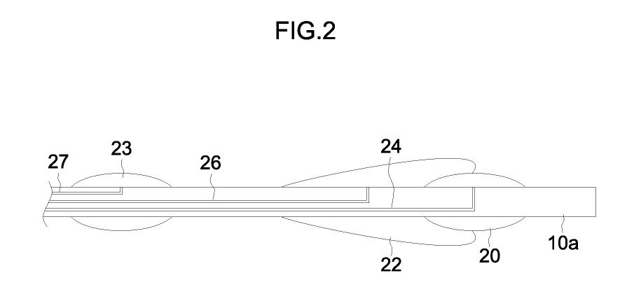

FIG. 2 is an enlarged sectional view along an axial direction (longitudinal direction) of the distal end portion 10a of the insertion portion 10 of the electronic endoscope 1 illustrated in FIG. 1.

As illustrated in FIG. 2, two balloons that are the driving balloon 20 and the locking balloon 22 are provided at the distal end portion 10a of the insertion portion 10 in sequence from the front side (distal end side) in the traveling direction.

Further, a holding balloon 23 is also provided. The holding balloon 23 can inflate or deflate to hold the position of the distal end portion 10a of the insertion portion 10 in a substantially center of the inside of the alimentary canal when the driving balloon 20 and the locking balloon 22 are not in contact with the inner wall of the alimentary canal.

The driving balloon 20, the locking balloon 22 and the holding balloon 23 are all formed from latex rubber which is inflatable and deflatable.

The locking balloon 22 is a balloon having an inflation characteristic such that the locking balloon 22 can inflate to make the locking balloon 22 contact with the inner wall surface of an alimentary canal and to lock the locking balloon 22 to the inner wall (to stop the locking balloon 22 in an engaged state).

Further, the driving balloon 20 and the locking balloon 22 preferably differ in shape from each other.

As illustrated in FIG. 2, the locking balloon 22 does not always have to cover the driving balloon 20 at the time of being deflated. The locking balloon 22 may preferably cover the driving balloon 20 at least when the locking balloon 22 is inflated and is locked to the inner wall of an alimentary canal.

Further, as illustrated in FIG. 2, a supply line 24 which communicates with the driving balloon 20 to supply a gas, a supply line 26 which communicates with the locking balloon 22 to supply a gas, and a supply line 27 which communicates with the holding balloon 23 to supply a gas are provided inside the distal end portion 10a. The supply lines 24, 26 and 27 are connected to the balloon control device 18 through the insides of the bending portion 10b, the flexible portion 10c and the code 14 (see FIG. 1).

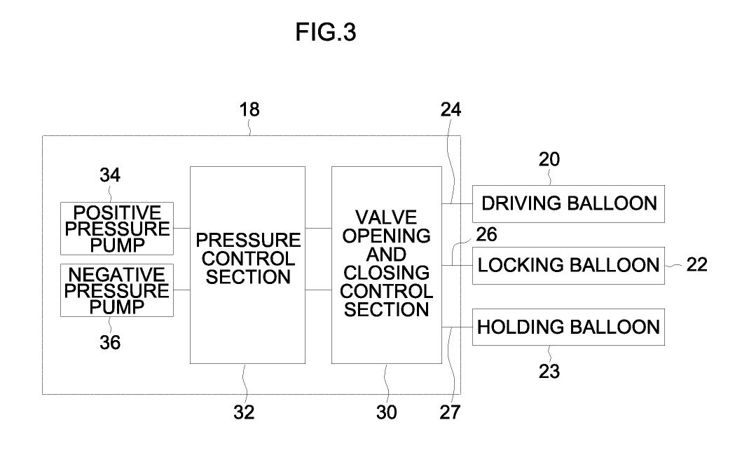

FIG. 3 is a block diagram of the balloon control device 18 which controls the pressures of the driving balloon 20, the locking balloon 22 and the holding balloon 23.

As illustrated in FIG. 3, the balloon control device 18 can control (regulate) the internal pressures of the driving balloon 20, the locking balloon 22 and the holding balloon 23 individually. A positive pressure pump (discharge pump) 34 and a negative pressure pump (suction pump) 36 are connected to the balloon control device 18 via a valve opening and closing control section 30 and a pressure control section 32.

For example, at the time of a normal traveling operation (propulsive operation), the valve opening and closing control section 30 controls opening and closing of a valve which is connected to each of the balloons. The pressure control section 32 controls the negative pressure pump (suction pump) 36 and the positive pressure pump (discharge pump) 34.

Hereinafter, the normal traveling operation (propulsive operation) will be described.

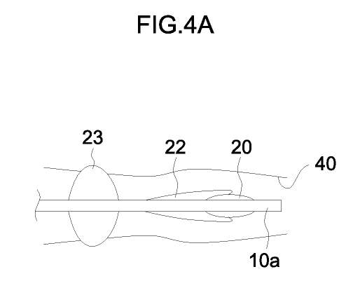

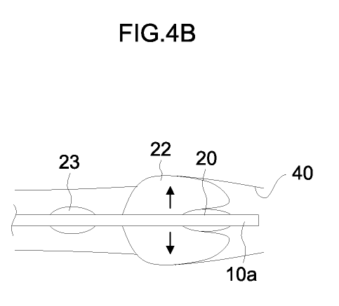

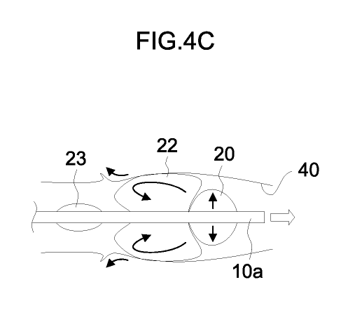

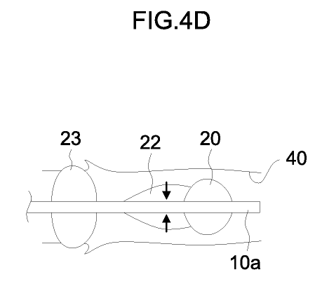

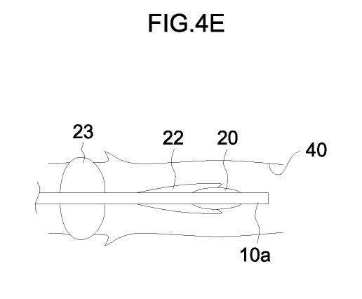

FIGS. 4A to 4E are enlarged sectional views along the axial direction (longitudinal direction) of the distal end portion 10a of the insertion portion 10 of the electronic endoscope 1 illustrating the state of inflation and deflation of each of the balloons at the time of the normal traveling operation.

First, in the state in which all the balloons are deflated, the distal end portion 10a of the electronic endoscope 1 is inserted into a measuring object (for example, a large intestine or the like) of a subject. As illustrated in FIG. 4A, in the state in which both the driving balloon 20 and the locking balloon 22 are deflated, the holding balloon 23 is inflated and locked to an intestinal wall 40.

Next, as illustrated in FIG. 4B, the locking balloon 22 is filled with gas to be inflated so that the locking balloon 22 is locked to the intestinal wall 40, and the holding balloon 23 is deflated. At this time, as illustrated in FIG. 4B, the locking balloon 22 inflates in such a manner as to cover the driving balloon 20 and is locked to the intestinal wall 40.

Next, in the state in which the locking balloon 22 is locked to the intestinal wall 40, the driving balloon 20 is filled with gas to be inflated. Thereupon, as illustrated in FIG. 4C, the driving balloon 20 gradually presses the part of the locking balloon 22 which covers the driving balloon 20. Next, the locking balloon 22 is pushed in such a manner that the surface of the locking balloon 22 is sequentially fed toward the rear side (proximal end side of the distal end portion 10a) in the traveling direction of the distal end portion 10a, or in such a manner that the surface of the locking balloon 22 is moved toward the rear side. Thereby, the locking balloon 22 gives a pressing force to the intestinal wall 40 toward the rear side in the traveling direction of the distal end portion 10a.

As a result, the locking balloon 22 is fed toward the rear side in the traveling direction of the distal end portion 10a while abutting on the intestinal wall 40 like a so-called Caterpillar (registered trademark in Japan). And, the intestinal wall 40 is pulled in to the rear side in the traveling direction of the distal end portion 10a. Accordingly, as illustrated by the white arrow in FIG. 4C, the distal end portion 10a of the electronic endoscope 1 propels to the front side (normal traveling) in the traveling direction relative to the intestinal wall 40.

Next, as illustrated in FIG. 4D, while the locking balloon 22 is deflated by sucking the gas, and the locking balloon 22 is alienated from the intestinal wall 40. The holding balloon 23 is inflated to be locked to the intestinal wall 40.

At this time, the intestinal wall 40 is assumed to keep the state in which it is pulled in to the rear side in the traveling direction of the distal end portion 10a even if the locking balloon 22 is alienated from the intestinal wall 40. If the intestinal wall 40 returns to the front side in the traveling direction of the distal end portion 10a when the locking balloon 22 is alienated from the intestinal wall 40, the locking balloon 22 can be deflated after the holding balloon 23 is locked to the intestinal wall 40.

Further, in the state of FIG. 4D, even if the locking balloon 22 is deflated, the locking balloon 22 can be in the state of abutting on the intestinal wall 40 without alienating from the intestinal wall 40 depending on the deflection amount of the intestinal wall 40, and it is suitable if only the locking balloon 22 does not apply a locking force to the intestinal wall 40.

Next, as illustrated in FIG. 4E, the driving balloon 20 is deflated by sucking the gas from the driving balloon 20. Thereby, the state becomes similar to that of FIG. 4A.

By repeating the above operation, the distal end portion 10a of the electronic endoscope 1 can be propelled toward the front side in the traveling direction (normal traveling operation).

Next, the fluid control method according to the presently disclosed subject matter will be described.

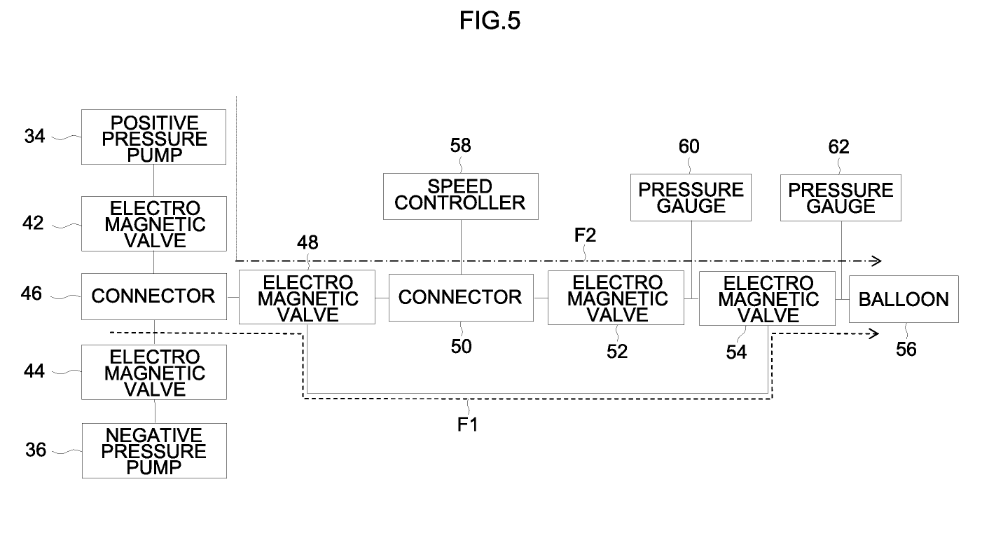

FIG. 5 illustrates a schematic configuration diagram of the first embodiment of a fluid control system of the presently disclosed subject matter.

As illustrated in FIG. 5, in the fluid control system of the first embodiment, the positive pressure pump 34 and the negative pressure pump 36 are connected to a connector 46 via electromagnetic valves 42 and 44 respectively. An electromagnetic valve 48, a connector 50, an electromagnetic valve 52, an electromagnetic valve 54 and a balloon 56 are connected following the connector 46. The balloon 56 corresponds to at least one of the drive balloon 20, locking balloon 22 and holding balloon 23.

Further, a speed controller 58 is connected to the connector 50. A pressure gauge 60 is connected to between the electromagnetic valve 52 and the electromagnetic valve 54. A pressure gauge 62 is connected to between the electromagnetic valve 54 and the balloon 56.

Here, the positive pressure pump 34 and the negative pressure pump 36 can be both the pumps with constant output powers. Since the output powers of the pumps (the positive pressure pump 34 and the negative pressure pump 36) are constant, the speed controller 58 can control (regulate) the pressure by reducing the flow rate. The speed controller 58 can reduce the pressure by releasing the pressure when the pressure in the system becomes too high. Further, the pressure gauge 60 is a pressure sensor with high precision which is used when the pressure of the balloon 56 is finely regulated to a predetermined pressure (target value) after the pressure of the balloon 56 reaches a certain pressure (threshold pressure). The pressure gauge 62 is a pressure sensor with a wide dynamic range which is used when the pressure in the balloon 56 increases to the certain pressure (i.e. before reaching the certain pressure). The presently disclosed subject matter intends to protect the pressure sensor (pressure gauge 60) with high precision.

The fluid control system of the first embodiment includes two conduit lines to protect the pressure sensor with high precision (pressure gauge 60). More specifically, they are a first flow F1 having a conduit line bypassing the pressure gauge 60 with high precision by the electromagnetic valve 48 and the electromagnetic valve 54 as illustrated by the broken line in FIG. 5, and a second flow F2 having a conduit line in which the speed controller 58 and the pressure gauge 60 with high precision are disposed between the electromagnetic valve 48 and the electromagnetic valve 54 as illustrated by the alternate long and short dash line. Switching of two conduit lines F1 and F2 is performed by the electromagnetic valve 48 and the electromagnetic valve 54.

When the balloon 56 is to be inflated, the electromagnetic valve 48 and the electromagnetic valve 54 are switched to the first flow F1 side first, and a large flow amount is passed to the balloon 56 at a high pressure and a high flow rate by the first flow F1 to inflate the balloon 56 quickly. At this time, the first flow F1 bypasses the conduit line to which the pressure gauge 60 with high precision is connected, and therefore, the pressure gauge 60 with high precision is protected so that a high pressure is not exerted thereon, and the fluid at a high pressure (20 to 100 kPa) is supplied to the balloon 56 at a high flow rate.

Subsequently, when the balloon 56 is inflated to some degree and the pressure measured by the pressure gauge 62 (approximately equal to the pressure in the balloon 56) reaches the certain pressure (threshold pressure), the electromagnetic valve 48 and the electromagnetic valve 54 are switched to the second flow F2 side. Then, pressure control of the balloon 56 is performed through the second flow F2. At this time, the pressure is measured by the pressure gauge 60 with a high precision which is disposed in the second flow F2 and pressure control of the balloon 56 is performed based on the measurement result of the pressure gauge 60. In the second flow F2, a part of the fluid is leaked from the speed controller 58, and the fluid at a low pressure/a small flow rate which is decompressed is supplied to the balloon 56. Like this, in the second flow F2, the pressure of the fluid is reduced by the speed controller 58 to be within the dynamic range of the pressure gauge 60 with high precision, and therefore, the pressure gauge 60 with high precision can be used without any problem.

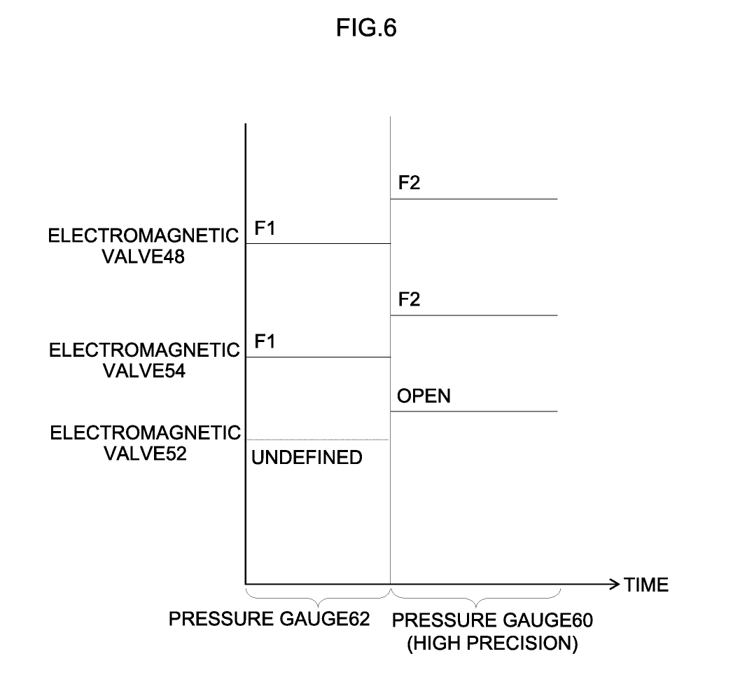

FIG. 6 is a timing chart of control of the electromagnetic valves 48, 52 and 54 when the balloon 56 is inflated.

When the balloon 56 is to be inflated, the electromagnetic valve 48 and the electromagnetic valve 54 are switched to the first flow F1 side first to communicate with the conduit line along the first flow F1 illustrated by the broken line in FIG. 5. At this time, the fluid does not flow into the conduit line to which the electromagnetic valve 52 is connected, and therefore, the electromagnetic valve 52 may be either opened or closed (undefined).

Thereby, the fluid at a high pressure/high flow rate is supplied to the balloon 56 from the positive pressure pump 34 through the conduit line of the first flow F1 illustrated by the broken line of FIG. 5. At this time, the pressure of the balloon 56 (fluid supplied to the balloon 56) is measured by the pressure gauge 62 with a wide dynamic range.

When it is confirmed that the pressure of the balloon 56 reaches the certain pressure (threshold pressure) based on the measurement result by the pressure gauge 62, the electromagnetic valve 48 and the electromagnetic valve 54 are switched to the second flow F2 side and the electromagnetic valve 54 is switched to the open side to communicate with the conduit line along the second flow illustrated by the alternate long and short dash line in FIG. 5.

Thereby, the fluid is decompressed to be within the dynamic range of the pressure gauge 60 by the speed controller 58, and is supplied to the balloon 56 through the conduit line of the second flow F2 illustrated by the alternate long and short dash line of FIG. 5. And then, the pressure in the balloon 56 is controlled to a predetermined pressure (target pressure). At this time, the pressure of the balloon 56 (approximately equal to the pressure of the fluid supplied to the balloon 56) is measured by the pressure gauge 60 with high precision.

As above, according to the present embodiment, selection (switching) of the conduit lines of the first flow F1 and the second flow F2 is realized by switching of the electromagnetic valves. In order to inflate/deflate the balloon 56 at a high speed, supply or exhaust of the fluid is performed through the first flow F1 at the initial stage of inflation/deflation. After a fixed time (after the fluid of a fixed volume is supplied), the conduit line is switched to the second flow F2, and pressure control with high precision is performed at a low flow rate.

The case of inflating the balloon 56 is described above. In the case of deflation, the first flow F1 is continued, and the negative pressure pump 36 may be stopped after a specified time (after the fluid of a specified volume is exhausted). Alternatively, the electromagnetic valve 48 is switched to the first flow F1 side whereas the electromagnetic valve 54 is set to the second flow F2 side, and the electromagnetic valve 52 is opened so that the balloon 56 is connected to the speed controller 58, whereby the fluid can be naturally exhausted from the position of the speed controller 58.

As above, in the present embodiment, by switching two conduit lines, the pressure sensor with high precision is protected, and the fluid at a high pressure/high flow rate can be supplied to the balloon 56 at a high speed. When only the conduit line of the second flow F2 is used without switching the conduits like this, the balloon control system is the same as the conventional balloon control system with a single conduit line.

When the electromagnetic valve 48 and the electromagnetic valve 54 are switched to the second flow F2 side after the electromagnetic valve 48 and the electromagnetic valve 54 are switched to the first flow F1 side to increase the pressure of the balloon 56 to a certain pressure, the electromagnetic valve 48 and the electromagnetic valve 54 are switched to the second flow F2 side and the electromagnetic valve 52 is closed to stop the flow of the fluid, whereby an accurate pressure of the balloon 56 may be measured with the pressure gauge 60.

When the pressure is higher than the predetermined pressure (target pressure) as a result of accurate pressure measurement of the balloon 56 by the pressure gauge 60, the electromagnetic valve 52 is opened to release the fluid from the speed controller 58 so that the pressure in the balloon 56 is reduced. At this time, the electromagnetic valve 52 may be opened in the state in which the electromagnetic valve 48 is switched to the first flow F1 side and the electromagnetic valve 54 is switched to the second flow F2 side at the same time. Thereby, the pressure in the balloon 56 can be regulated to a smaller value.

Next, a second embodiment of the fluid control system according to the presently disclosed subject matter will be described.

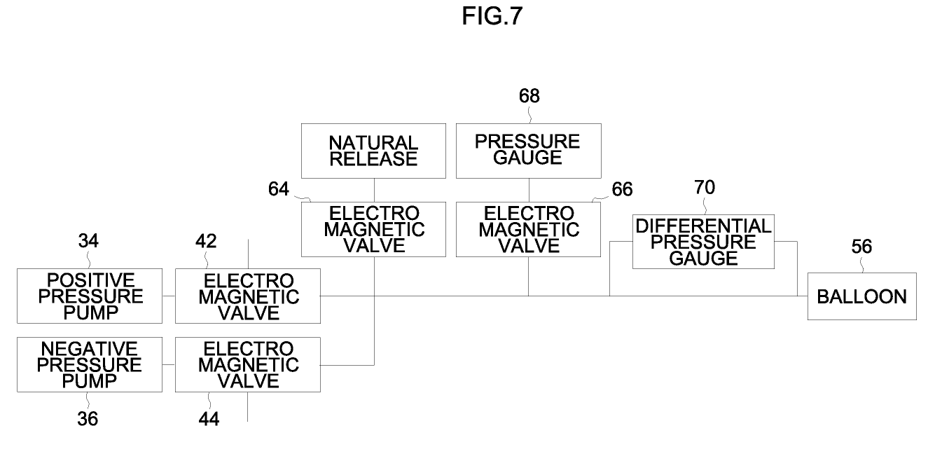

FIG. 7 illustrates a schematic configuration of the second embodiment of the fluid control system of the presently disclosed subject matter.

As illustrated in FIG. 7, in the fluid control system of the second embodiment, the positive pressure pump 34 and the negative pressure pump 36 are connected to a main conduit line which is connected to the balloon 56 via the electromagnetic valves 42 and 44 respectively. And, an electromagnetic valve 64 which is for naturally releasing the main conduit line, a pressure gauge 68 via an electromagnetic valve 66, and a differential pressure gauge 70 disposed in parallel with the main conduit line are connected to a middle region of the main conduit line. The balloon 56 corresponds to at least of the driving balloon 20, the locking balloon 22 and the holding balloon 23.

As in the first embodiment, the positive pressure pump 34 and the negative pressure pump 36 are the pumps with constant output powers. The pressure regulation of them is performed by natural release via the electromagnetic valve 64. Further, the pressure gauge 68 is a pressure sensor with high precision which is used when the pressure of the balloon 56 is finely regulated to a predetermined pressure (target pressure) after the pressure of the balloon 56 reaches a certain pressure (threshold pressure) similarly to the pressure gauge 60 of the first embodiment.

The differential pressure gauge 70 can measure the pressure difference between the two points in the main conduit line to which the differential pressure gauge 70 is attached. The flow rate of the fluid which passes through the main conduit line is calculated based on the measured pressure difference. Thereby, the total flow amount of the fluid which is supplied to the balloon 56 is determined.

Pressure control in the case of inflating the balloon 56 in the present embodiment will be described.

When the balloon 56 is inflated, the electromagnetic valve 64 is closed first, and the electromagnetic valve 66 is closed, whereby the pressure gauge 68 with high precision is alienated from the main conduit line. Thereby, the pressure gauge 68 with high precision is protected from a high pressure.

In the state in which natural release and the pressure gauge 68 are alienated from the main conduit line by the electromagnetic valves 64 and 66, the electromagnetic valve 42 is opened. Then, the fluid is supplied to the balloon 56 from the positive pressure pump 34 through the main conduit line.

Subsequently, based on the differential pressure between certain two points of the main conduit line measured by the differential pressure gauge 70, the flow rate of the fluid which is supplied to the balloon 56 through the main conduit line in a predetermined time period is calculated. By integrating the flow rate in the predetermined time period, the total quantity of the fluid supplied to the balloon 56 is obtained. The above calculation is performed by the pressure control section 32 in the balloon control device 18. A predetermined quantity of fluid is supplied into the balloon 56, and when it is determined that the balloon 56 is inflated to a certain degree, supply of the fluid to the balloon 56 is stopped.

Thereafter, when the pressure of the balloon 56 is regulated while a very small quantity of fluid is supplied to the balloon 56, the electromagnetic valve 64 and the electromagnetic valve 66 are opened, and the natural release and the pressure gauge 68 are connected to the main conduit line. Thereby, a fixed high pressure of the positive pressure pump 34 is reduced by natural release, and the fluid is supplied to the balloon 56 in the state in which the flow rate is reduced. Further, the pressure of the fluid supplied to the balloon 56 is measured through the main conduit line with the pressure gauge 68 with high precision, and pressure control is performed.

As above, in the present embodiment, the pressure gauge 68 with high precision is protected from high pressure by properly using the two kinds of systems that are the system which supplies the fluid at a high pressure through the main conduit line which connects the positive pressure pump 34 and the balloon 56 by alienating natural release and the pressure gauge 68 with high precision from the main conduit line, and the system which connects natural release and the pressure gauge 68 with high precision to the main conduit line and supplies the fluid at a very small flow rate.

Next, a third embodiment of the fluid control system according to the presently disclosed subject matter will be described.

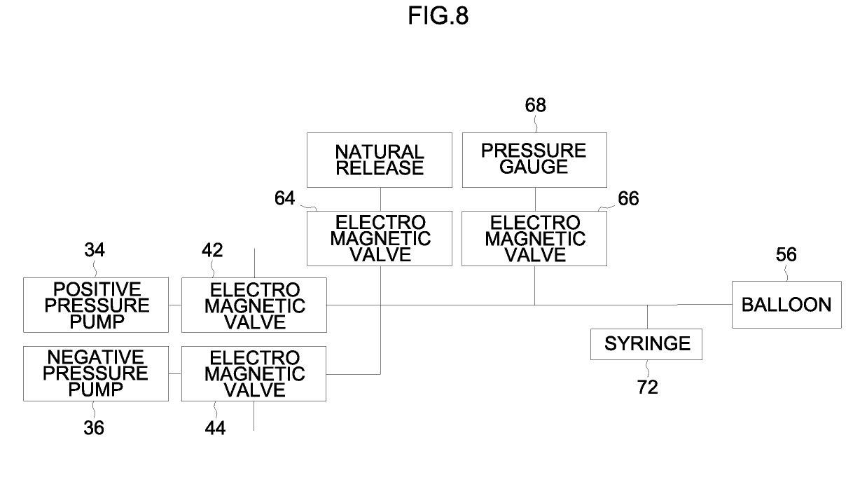

FIG. 8 is a schematic configuration diagram of the third embodiment of the fluid control system of the presently disclosed subject matter.

As illustrated in FIG. 8, in the present embodiment, a syringe 72 is connected to the main conduit line instead of the differential pressure gauge 70 in the second embodiment.

The differential pressure gauge 70 in the second embodiment is for calculating the flow rate of the fluid which is ultimately supplied to the balloon 56. The syringe 72 in the present embodiment is for supplying a fixed amount of fluid to the balloon 56 in a stroke.

Like this, in the present embodiment, when the balloon 56 is inflated, a fixed amount of fluid is supplied to the balloon 56 at a stroke by the syringe 72 first. Therefore, the pressure gauge (with a wide dynamic range) and the flowmeter which measure the pressure and the flow rate of the fluid at a high pressure which is supplied to the balloon 56 first do not have to be installed as in the above described first embodiment and second embodiment. Further, when the fluid at a high pressure is supplied to the balloon 56 by the syringe 72, the pressure gauge 68 with high precision is alienated from the main conduit line, and is protected from a high pressure.

Thereafter, when the pressure of the balloon 56 is regulated while the fluid is supplied to the balloon 56 at a very small flow rate, natural release and the pressure gauge 68 are connected to the main conduit line by opening the electromagnetic valve 64 and the electromagnetic valve 66. Thereby, a constant high pressure of the positive pressure pump 34 is reduced by natural release, and the fluid is supplied to the balloon 56 in the state in which the flow rate is reduced. Further, the pressure of the fluid which is supplied to the balloon 56 through the main conduit line is measured with the pressure gauge 68 with high precision, and pressure control is performed.

When the balloon 56 is deflated, a fixed amount of the fluid is sucked by the syringe 72 at a stroke.

As above, in the present embodiment, the pressure gauge 68 with high precision is protected from a high pressure by properly using the two kinds of systems that are the system which supplies the fluid at a high pressure from the main conduit line which connects the positive pressure pump 34 and the balloon 56 by alienating natural release and the pressure gauge 68 with high precision from the main conduit line, and the system which connects natural release and the pressure gauge 68 with high precision to the main conduit line and supplies the fluid at a very small flow rate.

Next, a fourth embodiment of the fluid control system according to the presently disclosed subject matter will be described.

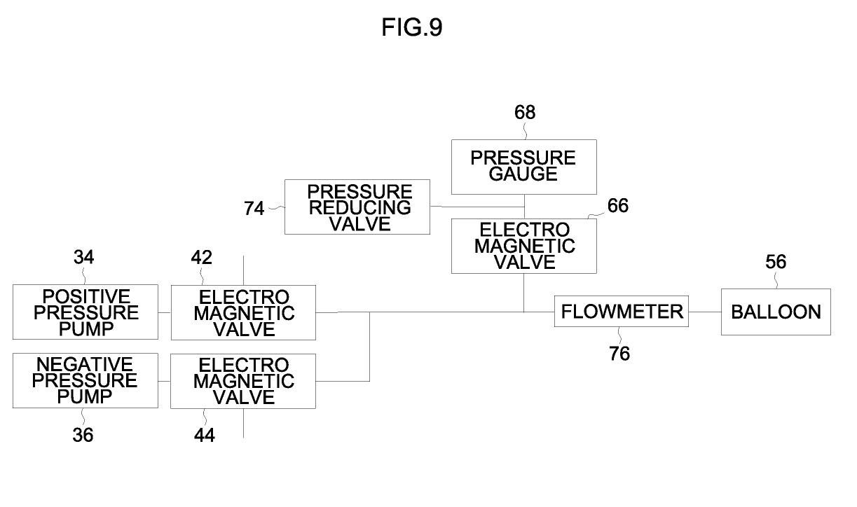

FIG. 9 is a schematic configuration diagram of the fourth embodiment of the fluid control system of the presently disclosed subject matter.

As illustrated in FIG. 9, in the present embodiment, the positive pressure pump 34, the negative pressure pump 36 and the balloon 56 are connected to the main conduit line, and the pressure gauge 68 with high precision is connected to the main conduit line via the electromagnetic valve 66. In the present embodiment, a pressure reducing valve 74 is provided between the electromagnetic valve 66 and the pressure gauge 68 instead of natural release (see FIGS. 7 and 8) via the electromagnetic valve 64, and a flowmeter 76 is installed instead of the differential pressure gauge 70 (see FIG. 7) and the syringe 72 (see FIG. 8).

In the present embodiment, when the balloon 56 is inflated, the electromagnetic valve 66 is closed, and the pressure gauge 68 with high precision is alienated from the main conduit line. Then, the electromagnetic valve 42 is opened, and the fluid at a high pressure is supplied to the balloon 56 from the positive pressure pump 34 through the main conduit line. At this time, the flow rate of the fluid which is supplied to the balloon 56 is measured with the flowmeter 76 which is installed in the main conduit line. Then, when a fixed amount is supplied to the balloon 56, the supply is stopped.

Thereafter, the pressure reducing valve 74 and the pressure gauge 68 are connected to the main conduit line by opening the electromagnetic valve 66. Then, the pressure of the balloon 56 is regulated while the fluid is supplied to the balloon 56 at a very low flow rate. Thereby, the fixed high pressure of the positive pressure pump 34 is reduced by the pressure reducing valve 74, and the fluid is supplied to the balloon 56 in the state in which the flow rate is reduced. Further, the pressure of the fluid which is supplied to the balloon 56 through the main conduit line is measured with the pressure gauge 68 with high precision, and pressure control is performed.

As above, in the present embodiment, the pressure gauge 68 with high precision is protected from a high pressure by properly using the two kinds of systems that are the system which supplies the fluid at a high pressure from the main conduit line which connects the positive pressure pump 34 and the balloon 56 by alienating the pressure gauge 68 with high precision from the main conduit line, and the system which connects the pressure gauge 68 with high precision and the pressure reducing valve 74 to the main conduit line and supplies the fluid at a very low flow rate.

The presently disclosed subject matter is not limited to only these embodiments. For example, as the device which determines whether a fixed amount is supplied when the fixed amount is supplied to the balloon 56 first when the balloon 56 is inflated, a pressure gauge with a wide dynamic range which measures the pressure of the balloon 56, a differential pressure gauge for calculating the flow rate from the differential pressure between the two points in the main conduit line, or a flowmeter which measures the flow rate of the fluid which directly passes through the main conduit line may be used.

Further, as the device for performing flow rate regulation after the fixed amount is supplied to the balloon 56, the leaking devices such as a speed controller, natural release and a pressure reducing valve may be combined for the pump with a constant output power, or the device for performing flow rate regulation may be realized by making the output power of the pump variable and reducing the output power by controlling the pump with a voltage/current. The pressure of the balloon 56 is regulated by the above described flow rate regulating device while the pressure is measured with the pressure gauge with high precision. When the output power of the pump is made variable and the pump is controlled with voltage/current, the flow rate is known by the voltage value, and therefore, a differential pressure gauge and a flowmeter may be eliminated.

In each of the aforementioned embodiments, the devices which determine whether the fixed amount is supplied and the devices for performing flow rate regulation may be combined by being properly replaced. For example, in the first, second and third embodiments, the pressure reducing valve may be combined in place of the speed controller and natural release. Further, for example, in the second embodiment and the fourth embodiment, the differential pressure gauge and the flowmeter may be replaced. Further, other than these examples, these devices may be combined by being replaced as much as possible.

As described above, according to each of the above described embodiments, the pressure gauge with high precision is protected from a high pressure by properly using the two kinds of systems that are the system which supplies the fluid at a high pressure from the main conduit line which connects the positive pressure pump and the balloon by alienating the pressure gauge with high precision from the main conduit line, and the system which connects the pressure gauge with high precision and the pressure regulating device to the main conduit line and supplies the fluid at a very low flow rate, and thereby, proper fluid control can be performed by using the pressure sensor with high precision while realizing a high flow velocity.

The fluid control device and the fluid control method, and the endoscope apparatus of the presently disclosed subject matter are described in detail above, but the presently disclosed subject matter is not limited to the above examples, and various improvements and modifications may be made within the range without departing from the gist of the presently disclosed subject matter as a matter of course.

What is claimed is:

1. A fluid control device which controls a flow rate of a fluid which is supplied or sucked to or from an inflation and deflation member included in an endoscope or an endoscope auxiliary tool, comprising:
a fluid flow generation device which generates a fluid flow to supply or suck the fluid to or from the inflation and deflation member;
a first pipe system which is connected to the inflation and deflation member, wherein the fluid flow of a predetermined flow rate generated by the fluid flow generation device is supplied to the first pipe system to supply or suck the fluid to or from the inflation and deflation member;
a second pipe system which is connected to the inflation and deflation member, wherein a fluid flow of a flow rate lower than the predetermined flow rate is supplied to the second pipe system to supply or suck the fluid to or from the inflation and deflation member;
a pipe system switching device which switches the first pipe system to the second pipe system when a predetermined amount of fluid is supplied or sucked to or from the inflation and deflation member through the first pipe system;
a pressure sensor which is included in the second pipe system, and measures a pressure in the second pipe system; and
a pressure control device which controls the pressure in the inflation and deflation device by using the second pipe system based on the pressure measured by the pressure sensor.

2. The fluid control device according to claim 1, further comprising:
a leaking device which regulates the flow rate of the fluid by leaking a part of the fluid to an outside; and
a main conduit line which allows the fluid flow generation device and the inflation and deflation member to communicate with each other,
wherein an output of the fluid flow generation device is constant,
the second pipe system is a conduit line which connects the leaking device and the pressure sensor to the main conduit line, and
the first pipe system is a conduit line which extends from the fluid flow generation device to the inflation and deflation member by passing a bypass conduit line which bypasses the leaking device and the pressure sensor, and includes a flow rate adjustment device for supplying or sucking a fixed amount of the fluid to or from the inflation and deflation member.

3. The fluid control device according to claim 1, further comprising:
a leaking device which regulates the flow rate of the fluid by leaking a part of the fluid to an outside; and
a main conduit line which allows the fluid flow generation device and the inflation and deflation member to communicate with each other, connects the leaking device and the pressure sensor to be capable of communicating with each other and being cut off from each other, and includes a flow rate adjustment device for supplying or sucking a constant amount of the fluid to or from the inflation and deflation member,
wherein the fluid flow generation device has a constant output,
the first pipe system is a conduit line for cutting off the leaking device and the pressure sensor from the main conduit line, and
the second pipe system is a conduit line which allows the leaking device and the pressure sensor to communicate with the main conduit line.

4. The fluid control device according to claim 2,
wherein the flow rate adjustment device is a flow rate integration detecting device which detects that the amount of the fluid which is supplied or sucked to or from the inflation and deflation member reaches a predetermined amount.

5. The fluid control device according to claim 3,
wherein the flow rate adjustment device is a flow rate integration detecting device which detects that the amount of the fluid which is supplied or sucked to or from the inflation and deflation member reaches a predetermined amount.

6. The fluid control device according to claim 4,
wherein the flow rate integration detecting device includes at least a pressure gauge.

7. The fluid control device according to claim 4,
wherein the flow rate integration detecting device includes at least a differential pressure gauge.

8. The fluid control device according to claim 4,
wherein the flow rate integration detecting device includes at least a flowmeter.

9. The fluid control device according to claim 2,
wherein the flow rate adjustment device is a syringe which supplies or sucks a constant amount of the fluid which is set in advance to or from the inflation and deflation member.

10. The fluid control device according to claim 3,
wherein the flow rate adjustment device is a syringe which supplies or sucks a constant amount of the fluid which is set in advance to or from the inflation and deflation member.

11. The fluid control device according to claim 2,
wherein the leaking device is a speed controller.

12. The fluid control device according to claim 2,
wherein the leaking device is a device for naturally releasing the main conduit line.

13. The fluid control device according to claim 2,
wherein the leaking device is a pressure reducing valve.

14. A fluid control method for controlling a flow rate of a fluid which is supplied or sucked to or from an inflation and deflation member included in an endoscope or an endoscope auxiliary tool by a fluid flow generation device, comprising the steps of:
generating a fluid flow to supply or suck the fluid to or from the inflation and deflation member;
supplying the fluid flow of a predetermined flow rate generated by the fluid flow generation device to a first pipe system connected to the inflation and deflation member, to supply or suck the fluid to or from the inflation and deflation member;
switching the first pipe system to a second pipe system when a predetermined amount of fluid is supplied or sucked to or from the inflation and deflation member through the first pipe system;
supplying a fluid flow of a flow rate lower than the predetermined flow rate to the second pipe system connected to the inflation and deflation member, to supply or suck the fluid to or from the inflation and deflation member; and
controlling the pressure in the inflation and deflation device by controlling the fluid flow in the second pipe system based on the pressure measured by a pressure sensor which measures a pressure in the second pipe system.

15. An endoscope apparatus, comprising a fluid control device according to claim 1.
